SEQUENCE LISTING (25 sequences)

SEQ ID NO: 1 - DNA, 28 bp, Artificial Sequence
  Primer APC-a5Xh
ccgctcgagatggctgcagcttcatatg

SEQ ID NO: 2 - DNA, 28 bp, Artificial Sequence
  Primer APC-a3Ba
cgggatccctgtggtcctcatttgtagc

SEQ ID NO: 3 - DNA, 30 bp, Artificial Sequence
  Primer APC-b5Xh
ccgctcgagcaaatcctaagagagaacaac

SEQ ID NO: 4 - DNA, 28 bp, Artificial Sequence
  Primer APC-b3Ba
cgggatcctggtccattatctttttcac

SEQ ID NO: 5 - DNA, 18 bp, Artificial Sequence
  Primer AV-a5
cttcatattagatgcctc

SEQ ID NO: 6 - DNA, 19 bp, Artificial Sequence
  Primer AV-a3
gctgatatatgtgctgtag

SEQ ID NO: 7 - DNA, 18 bp, Artificial Sequence
  Primer AV-b5
gtgctttgaatgaatgag

SEQ ID NO: 8 - DNA, 18 bp, Artificial Sequence
  Primer AV-b3
cctacagaaggcagaagt

SEQ ID NO: 9 - DNA, 28 bp, Artificial Sequence
  Primer BRCA1-a5Xh
ccgctcgagagttcattggaacagaaag

SEQ ID NO: 10 - DNA, 28 bp, Artificial Sequence
  Primer BRCA1-a3H
cccaagcttgatacttttctggatgcct

SEQ ID NO: 11 - DNA, 28 bp, Artificial Sequence
  Primer BRCA1-b5Xh
ccgctcgaggctgcttgtgaattttctg

SEQ ID NO: 12 - DNA, 28 bp, Artificial Sequence
  Primer BRCA1-b3H
cccaagcttttgaatccatgctttgctc

SEQ ID NO: 13 - DNA, 27 bp, Artificial Sequence
  Primer BRCA1-c5Xh
ccgctcgagatgaggcatcagtctgaa

SEQ ID NO: 14 - DNA, 28 bp, Artificial Sequence
  Primer BRCA1-c3H
cccaagcttggctgtgggggatctgggg

SEQ ID NO: 15 - DNA, 18 bp, Artificial Sequence
  Primer BV-a5
cagacagatgggacactc

SEQ ID NO: 16 - DNA, 21 bp, Artificial Sequence
  Primer BV-a3
gaattttctgagacggatgta

SEQ ID NO: 17 - DNA, 18 bp, Artificial Sequence
  Primer BV-b5
cacatgcaagtttgaaac

SEQ ID NO: 18 - DNA, 18 bp, Artificial Sequence
  Primer BV-b3
ttcttgattggttcttcc

SEQ ID NO: 19 - DNA, 21 bp, Artificial Sequence
  Primer BV-c5
acctaagtttgaatccatgct

SEQ ID NO: 20 - DNA, 18 bp, Artificial Sequence
  Primer BV-c3
acccgagagtgggtgttg

SEQ ID NO: 21 - DNA, 21 bp, Artificial Sequence
  Primer APC-b5
caaatcctaagagagaacaac

SEQ ID NO: 22 - DNA, 21 bp, Artificial Sequence
  Primer APC-b3
gtccattatctttttcacacg

SEQ ID NO: 23 - DNA, 19 bp, Artificial Sequence
  Primer Seq-F
tcatctttgtcatcagctg

SEQ ID NO: 24 - DNA, 18 bp, Artificial Sequence
  Primer Seq-R
agataaactagaaccctg

SEQ ID NO: 25 - DNA, 18 bp, Artificial Sequence
  Primer BR-Seq
tatttctgggtgacccag

(19) United States
(12) Patent Application Publication
(10) Pub. No.: US 2003/0064365 A1
(43) Pub. Date: Apr. 3, 2003

(54) Gap vector for E. coli stop codon assay and method for detecting heterozygous mutation using the same

(76) Inventors: Young-Ho Moon, Kyonggi-do (KR); Hye-Jung Nam, Seoul (KR); Dong-Hwan Kim, Kyonggi-do (KR); Hyun-Pil Cho, Kyonggi-do (KR); Su-Mi Han, Inchon-shi (KR); Mi-Uk Chin, Seoul (KR); Seg-Ho Choi, Kyonggi-do (KR); Su-Yun Kim, Kyonggi-do (KR); Sang-Yong Choi, Seoul (KR); Byung-Joo Song, Seoul (KR); Eun-Ryoung Kim, Seoul (KR); Hyo-Jong Kim, Seoul (KR); Il-Soo Kim, Seoul (KR)

Correspondence Address: NIXON & VANDERHYE P.C., 8th Floor, 1100 North Glebe Road, Arlington, VA, 22201-4714, US

(21) Appl. No.: 09/888,625

(22) Filed: Jun. 26, 2001

(51) Int. Cl. C12Q 1/68; C12P 19/34

(52) U.S. Cl. 435/6; 435/91.2; 435/448

(57) ABSTRACT

The present invention provides a method for detecting heterozygous mutation using E. coli stop codon assay. The present invention further provides a gap vector used in the E. coli stop codon assay. According to this invention, the heterozygous mutation in certain gene, e.g. APC gene or BRCA1 gene, may be detected in simple and rapid manner, for example, visual observation on colonies.

### BACKGROUND OF THE INVENTION

1. Field of the Invention

The present invention relates to gap vectors for E. coli stop codon assay and methods for detecting heterozygous mutation using the same. More particularly, this invention relates to gap vectors for E. coli stop codon assay comprising exon(s) of a gene of interest which is susceptible to truncating mutation and methods for detecting heterozygous mutation using the same.

2. Description of the Related Art

The methods for detection or screening of gene mutation, having been known in the art, include SSCP (single strand conformation polymorphism) using PCR (polymerase chain reaction), RPA (RNase protection assay), DGGE (denaturing gradient gel electrophoresis), mismatch detection non-isotopic RNase cleavage assay, CFLP (cleavage fragment length polymorphism) and PTT (protein truncation test). The results from the methods, in general, are confirmed by DNA sequencing to identify base changes.

The above methods for detection of gene mutation have disadvantages as well as advantages and the selection of a suitable method depends on the purpose of the research and the object to be studied. For example, SSCP, RPA or DGGE are usually employed for detection of mutations in APC gene (Kinzler K W. Nilbert M C, Su LK, et al., Science 253(5020):661-665(1991)) or BRCA1 gene (Miki Y. et al., Science 266(5182):66-71(1994)) which are responsible for genetic breast cancer, ovarian cancer and familial adenomatous polyposis (FAP) which develops to colorectal cancer. However, the methods have shortcomings: (a) restricted region of gene to be screened; and (b) no clear distinction between mutated and normal DNAs, which depends on certain conditions. Furthermore, if the gene to be tested is large in size and has a wide distribution of mutated regions throughout itself, the amount of testing is relatively increased to a great extent, which may be a critical obstacle in performing the assay. In studying genes associated with cancer, the identification of novel mutations in tumor suppressor genes has a difficulty, because the genes are generally large in size and mutations in the genes are heterozygous. Therefore, to carry DNA sequencing for the identification of mutations is a troublesome experimentation and where assay is performed for many samples, the cost therefor may be significantly increased.

The in vivo functions of a large majority of cancer-related genes are not well known and many of the genes are disrupted mainly by truncating mutations. Virtually all mutations in APC gene and more than 80% of mutations in BRCA1 gene are ascribed to nonsense or frameshift mutations, a type of truncating mutations, leading to generating stop codon. Hitherto, for screening of the nonsense mutations, PTT is usually employed, in vitro detecting truncated proteins generated by nonsense or frameshift mutations. The method, however, requires significant technical skill and gives no definite results, thereby rendering it very ineffective. In addition, where the method is performed for many samples, the cost therefor may be significantly increased.

To be free from the above mentioned shortcomings, Ishioka et al. has provided stop codon assay for detection of heterozygous truncating mutations using yeast cells (Ishiok, C. et al., Proc. Natl. Acad. Sci. USA., 94(6):2449-2453(1997)), which is incorporated by reference into this application. Ishioka et al. has screened heterozygous truncating mutations in APC and BRCA1 genes by a method using homologous recombination in yeast. However, the method has recognized some drawbacks; the method employs yeast cells, which has difficulty in transformation and requires significant time and cost.

Therefore, there remains a need in the art for a more effective method for detection of heterozygous truncating mutations, capable of reducing time and cost required.

Throughout this application, various patents and publications are referenced and citations are provided in parentheses. The disclosure of these patents and publications in their entities are hereby incorporated by references into this application in order to more fully describe this invention and the state of the art to which this invention pertains.

### SUMMARY OF THE INVENTION

In one aspect of this invention, there is provided a method for detecting heterozygous mutation on a gene of interest, which comprises the steps of: (a) amplifying exon(s) of the gene of interest which is susceptible to truncating mutation; (b) cloning the amplified exon(s) into plasmid with a low copy number for E. coli; (c) performing PCR with the plasmid as template carrying the exon(s) and a set of primers designed to produce a gap vector having nucleotides of 50-200 bp at 5′ end and 3′ end thereof, respectively, corresponding to nucleotides at 5′ end and 3′ end of the amplified exon(s), respectively; (d) isolating RNA or gDNA from specimen and amplifying RNA or gDNA fragment by RT-PCR or PCR corresponding to the amplified exon(s); (e) cotransforming E. coli with the gap vector and the amplified DNA fragment of (d); and (f) determining the occurrence of gap repair of the gap vector of (e), wherein the gap repair is ascribed to homologous recombination between nucleotides at 5′ end and 3′ end of the gap vector and the cotransformed DNA fragment.

In one embodiment, the gene of interest is APC gene or BRCA1 gene.

In an alternative embodiment, the gene of interest is APC gene and the set of primers to produce the gap vector are oligonucleotides consisting of the nucleotide sequence represented by SEQ ID NO:1 and SEQ ID NO:2 designed for amplifying exon 1 to exon 14 of APC gene.

In the preferred embodiments, the gene of interest is APC gene and the set of primers to produce the gap vector are oligonucleotides consisting of the nucleotide sequence represented by SEQ ID NO:3 and SEQ ID NO:4 designed for amplifying exon 15 of APC gene

In an alternative embodiment, the gene of interest is BRCA1 gene and the set of primers to produce the gap vector are oligonucleotides consisting of the nucleotide sequence represented by SEQ ID NO:9 and SEQ ID NO:10 designed for amplifying exon 2 to exon 10 of BRCA1 gene.

In the preferred embodiments, the gene of interest is BRCA1 gene and the set of primers to produce the gap vector are oligonucleotides consisting of the nucleotide sequence represented by SEQ ID NO:11 and SEQ ID NO:12 designed for amplifying exon 11 of BRCA1 gene.

In the preferred embodiments, the gene of interest is BRCA1 gene and the set of primers to produce the gap vector are oligonucleotides consisting of the nucleotide sequence represented by SEQ ID NO:13 and SEQ ID NO:14 designed for amplifying exon 12 to exon 24 of BRCA1 gene.

In the preferred embodiments, the gap vector carries lacZ gene as selective marker to determine the occurrence of the gap repair.

In the preferred embodiments, the step (f) is performed by incubating the cotransformed E. coli in medium containing X-gal and IPTG (isopropyl-β-D-thiogalactoside).

In the preferred embodiments, the plasmid with a low copy number has a replication origin derived from F plasmid, most preferably, ori-2.

In another aspect of this invention, there is provided a gap vector for E. coli stop codon assay comprising: (a) DNA fragment of a gene of interest located at 5′ end of the gap vector in which the gene of interest is susceptible to truncating mutation; (b) DNA fragment of the gene of interest located at 3′ end of the gap vector in which the DNA fragment has a different sequence from the DNA fragment of (a); (c) lacZ gene downstream of the DNA fragment of (a); (d) lac operator and promoter upstream of the DNA fragment of (b) to control the expression of the DNA fragments and lacZ gene; and (e) replication origin ensuring the copy number of the gap vector to be 1-1.5; in which a gap exists between the DNA fragments (a) and (b), whereby a homologous recombination occurs between the DNA fragments and a gene of interest to lead to gap repair of the gap vector.

In the preferred embodiments, the replication origin contained in the gap vector is derived from F plasmid, most preferably, ori-2.

In the preferred embodiments, the DNA fragments of gene of interest located at 5′ end and 3′ end of the gap vector the DNA fragments are 50-200 bp in size.

In the preferred embodiments, the gene of interest is APC gene or BRCA1 gene

In still another aspect of this invention, there is provided a gap vector for E. coli stop codon assay constructed by a process comprising the steps of: (a) amplifying exon(s) of a gene of interest which is susceptible to truncating mutation; (b) cloning the amplified exon(s) in plasmid with a low copy number for E. coli; and (c) performing PCR with the plasmid carrying the exon(s) as template and a set of primers to produce a gap vector having nucleotides of 50-200 bp at 5′ end and 3′ end thereof, respectively, corresponding to nucleotides at 5′ end and 3′ end of the amplified exon(s), respectively; in which a gap is generated between the nucleotides located at 5′ end and at 3′ end.

In the preferred embodiments, the plasmid for E. coli has a replication origin derived from F plasmid, most preferably, ori-2.

Accordingly, it is an object of this invention to provide a method for detecting heterozygous mutation using E. coli stop codon assay.

It is another object of this invention to provide a gap vector for E. coli stop codon employed in detecting heterozygous mutation.

Other objects and advantages of the present invention will become apparent from the detailed description to follow taken in conjugation with the appended claims and drawings.

### BRIEF DESCRIPTION OF THE DRAWINGS

FIG. 1 is a drawing representing briefly a method of this invention using E. coli stop codon assay;

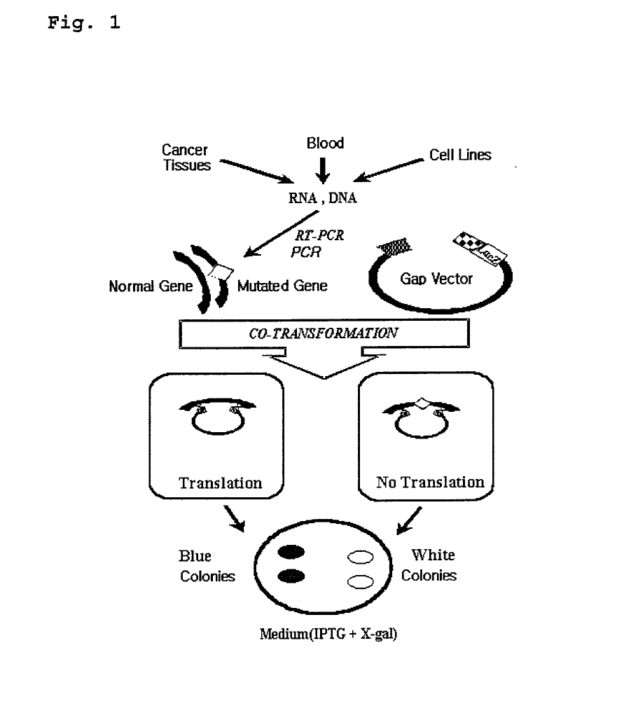

FIG. 2 is a genetic map of the vector pZC320 used in preparing the gap vector according to one embodiment of this invention;

FIG. 3 is a photograph of agarose gel electrophoresis of plasmid pAPC-a digested with restriction enzymes;

FIG. 4 is a photograph of agarose gel electrophoresis of plasmid pAPC-b digested with restriction enzymes;

FIG. 5 is composed of genetic maps of gap vectors AV-a and AV-b constructed according to one embodiment of this invention;

FIG. 6 is composed of photographs of agarose gel electrophoresis of plasmids pBRCA1-a, pBRCA1-b and pBRCA1-c digested with restriction enzymes;

FIG. 7 is composed of genetic maps of gap vectors BV-a, BV-b and BV-c constructed according to one embodiment of this invention;

FIG. 8 is composed of photographs of agarose gel electrophoresis of gap vectors AV-a, AV-b, BV-a, BV-b and BV-c of this invention;

FIG. 9 is composed of photographs of agarose gel electrophoresis of PCR-amplified fragments APC-a and APC-b derived from several different samples;

FIG. 10 is a photograph of agarose gel electrophoresis of PCR-amplified fragment APC-b derived from Gardener's Syndrome (GS) family;

FIG. 11 is composed of photographs of agarose gel electrophoresis of plasmids digested with restriction enzymes, which are derived from blue or white colonies formed from E. coli stop codon assay on APC gene according to this invention;

FIG. 12 contains photographs showing the results of DNA sequencing for confirming the reliability of the results on detection of mutations in APC gene derived from Gardener's Syndrome (GS) family members according to this invention;

FIG. 13 contains photographs showing the results of DNA sequencing of APC alleles isolated as E. coli stop codon assay of this invention;

FIG. 14 contains photographs of agarose gel electrophoresis of PCR-amplified fragments BRCA1-a, BRCA1-b and BRCA1-c derived from several different samples; and

FIG. 15 contains photographs showing the results of DNA sequencing of BRCA1 alleles isolated as E. coli stop codon assay of this invention.

### DETAILED DESCRIPTION OF THE INVENTION

The present invention relates to a method for detecting heterozygous mutation in several different genes using E. coli stop codon (hereinafter referred at as “ESC”) assay.

The present method is described in more detail as set forth hereunder:

I. Cloning of Exon Sequence

Exon region (s) of a certain gene, which is susceptible to truncating mutation, in other words, which shows a high frequency of truncating mutation, is (are) amplified by PCR and then cloned into plasmid with a low copy number for E. coli. For example, the gene which is susceptible to truncating mutation includes, but not limited to, APC and BRCA1 genes. Preferably, in case of APC gene, exons 1-14 corresponding to nucleotides 19-1977 or exon 15 corresponding to nucleotides 1978-5266 are amplified by RT-PCR or PCR and in case of BRCA1 gene, exons 2-10 corresponding to nucleotides 104-904,exon 11 corresponding to nucleotides 789-4217 and exons 12-24 corresponding to nucleotides 4089-5704 are amplified by RT-PCR or PCR. Amplifying a long entire gene is very difficult and is error-prone, if possible. Thus, using fragments of an entire gene in this invention is preferable in light of accuracy and reproducibility of the assay.

It is preferable that the amplified exon sequences further contain recognition sites directed to BamHI, EcoRI, HindIII, XbaI, XhoI or SacI for subsequent cloning. The recognition sites may be inserted into the amplified exon by way of primers of PCR containing the sites.

The amplified exon sequences are then cloned into plasmid for E. coli. To use plasmid with very a low copy number is vital in this invention, permitting effective separation of heterozygote. The plasmid useful in this invention includes any of the plasmids with a low copy number for E. coli, 1-5 copies, preferably 1-3 copies, more preferably 1-2 copies and the most preferably 1-1.5 copies. The copy number of plasmid depends mainly on replication origin. For example, a replication origin derived from F plasmid renders plasmid containing the origin sequence to be stringent (Kahn, M. et al., Methods in Enzymology, 68:268(1979)).

For the purpose of the above, the plasmid employed in this invention carrying a replication origin derived from F, pSC101 (Stoker, N. G. et al., Gene, 18:335(1982)) or pOU71 (Larsen, J. E. L. et al., Gene, 28:45(1984)) is preferred. The most preferred plasmid is one carrying a replication origin derived from F plsmid, having 1-1.4 copies per E. coli cell, which ensures it most suitable for a separation of alleles.

The cloning vector used in this invention carries preferably selective marker gene, for example, lacZ encoding β-galactosidase. LacZ product permits screening of the occurrence of gap repair by incubation of transformants on solid medium containing X-gal and IPTG, thereby enabling visual observation.

Furthermore, preferably, the cloning vector carries sop gene enabling stable plasmid maintenance in the absence of selection.

II. Construction of Gap Vector

The plasmids containing exons from stage I are used as template and the primers, which are designed for producing gap vector having nucleotides of 50-200 bp at 5′ end and 3′ end, respectively, corresponding to nucleotides at 5′ end and 3′ end of the cloned exons, are used in PCR for preparing gap vector for ESC assay. The length of nucleotides at 5′ and 3′ ends of gap vector is very important. If the length is less than 50 bp, a homologous recombination following cotransformation may not be expected; in the case of more than 200 bp, the length of gene to be assayed may be subject to restriction.

As a result of the PCR, a desired gap vector is given comprising: DNA sequences necessary for function as plasmid with a low copy number for E. coli and nucleotides of 50-200 bp at 5′ end and 3′ end, respectively, corresponding to nucleotides at 5′ end and 3′ end of the amplified exons, thereby generating a gap between the nucleotides at 5′ and 3′ ends.

The gap vector according to this invention, is a linear DNA fragment rather than circular plasmid due to gap mentioned above, while comprising all DNA sequences necessary for replication in E. coli. Thus, gap vector introduced into E. coli cannot be replicated unless the gap is repaired through homologous recombination between the ends of gap vector and DNA sequences corresponding to gap. This fact prevents the inventors from depositing the gap vector and the microorganism according to this invention in a Depository Authority.

III. Amplification of Exon Sequence

PCR amplification is performed using, as template, RNA or gDNA isolated from a variety of specimens to be assayed, thus amplifying DNA fragment corresponding to exon region amplified in stage I. The specimen is determined to have mutation, particularly heterozygous mutation, in certain gene, i.e. APC or BRCA1 genes. The template is obtained as RNA or gDNA from individual blood, cells or established cell lines.

The PCR uses primers designed for selective amplification of DNA fragments corresponding to DNA fragments amplified in stage I. For example, in case of APC gene, primers for PCR amplification of DNA fragments corresponding to exons 1-14 or exon 15, in case of BRCA1 gene, primers for PCR amplification of DNA fragments corresponding to exons 2-10, exon 11 or exons 12-24 are used desirably. The PCR-amplified DNA fragments are then purified by way of conventional method, i.e. agarose gel electrophoresis.

IV. Cotransformation

E. coli is cotransformated with the gap vector of stage II and DNA fragments of stage III. Thereafter, the gap vector is repaired in E. coli through homologous recombination between DNA fragments of 50-200 bp at 5′ and 3′ ends of the gap vector and the DNA fragments of stage III, finally rendering to be replicable plasmid. The gene coding for selective marker, e.g. β-galactosidase, on the replicable plasmid is translated and thus enables to determine the existence of mutation.

In the preferred embodiments in which lacZ gene is located downstream of insert sequence, the homologous recombination between the gap vector and DNA fragment without mutation allows to gap repair, i.e., undergo recircularization of the vector and subsequently express β-galactosidase encoded by lacZ, finally forming blue-colored colonies on medium containing X-gal and IPTG. In contrast to this, the homologous recombination between the gap vector and DNA fragment with heterozygous mutation cannot repair the gap in the vector and express β-galactosidase, finally forming white-colored colonies.

Therefore, according to the preferred embodiments, the heterozygous mutation in certain gene is detected in simple and rapid manner: visual observation on medium containing X-gal and IPTG.

The following specific examples are intended to be illustrative of the invention and should not be construed as limiting the scope of the invention as defined by appended claims.

### EXAMPLE I-1

### Construction of Gap Vector for ESC Screening of APC Gene Mutation

I-1: APC Gene Cloning in Vector with a low Copy Number for E. Coli

To construct plasmid employed as a template in PCR which is carried out in the construction of gap vector for ESC screening of APC gene, two fragments spanning nucleotides 19-1977 and nucleotides 1978-5266 of APC gene (Genebank Accession No. M74088), corresponding to its exons 1-14 and exon 15, respectively, were cloned into a plasmid with a low copy number for E. coli as follows:

The primers APC-a5Xh (see SEQ ID NO: 1) and APC-a3Ba (see SEQ ID NO: 2) were synthesized for cloning of nucleotides 19-1977 of APC gene corresponding to its exons 1-14 in a plasmid with a low copy number for E. coli. In addition, total cellular RNA was isolated from HCT116 cell line (ATCC, CCL-247. USA) and amplified by RT-PCR as follows: The total cellular RNA from the above cell line as template isolated using TriZOL (GIBCO-BRL, USA) 400 ng, 25 mM MgCl₂ 4 μl, 10X RNA PCR buffer (100 mM Tris-HCl (pH 8.3) and 500 mM KCl) 2 μl, 10 mM dNTP solution 2 μl, 40 units/μl RNase inhibitor 0.5 μl, 5 units/μl AMV reverse transcriptase XL 1 μl and oligo d(T)-adaptor primer 1 μl was adjusted to the final volume of 20 μl with DW and well mixed. Thereafter, with resulting reaction, cDNA was synthesized according to the temperature conditions: 30° C. for 10 min, 42° C. for 40 min and 99° C. for 5 min, 1 cycle. For amplifying APC gene, to the resulting cDNA of 20 μl, 25 mM MgCl₂ 6 μl, 10X LA PCR buffer II 8 μl, sterilized water 65.1 μl, TaKaRa Ex Taq 0.5 μl, and 10 pmol primers APC-a5Xh and APC-a3Ba of 2 μl, respectively were added, completely mixed, subject to denaturation at 94° C. for 5 min and then to PCR amplification for 30 cycles according to temperature parameters as follows: 94° C. for 30 sec, 56° C. for 30 sec and 72° C. for 1 min. The PCR product was identified by electrophoresis with 1.5% agarose gel containing ethidium bromide for staining and was digested with XhoI and BamHI, followed by purification using GENECLEAN II kit (Bio101, USA). The purified fragment was denoted as “APC-a”.

The primers APC-b5Xh (see SEQ ID NO: 3) and APC-b3Ba (see SEQ ID NO: 4) were synthesized for cloning of nucleotides 1978-5266 of APC gene corresponding to its exon 15 in a plasmid with a low copy number for E. coli. In addition, gDNA was isolated from normal individuals and amplified by PCR as follows: The gDNA as template 1 μg, 10X reaction buffer (100 mM Tris-HCl(pH 8.3), 500 mM KCl and 15 mM MgCl₂) 5 μl, primers APC-b5Xh and APC-b3Ba of 10 pmol, respectively and Ex. Taq polymerse (Takara, Japan) 2.5 unit were adjusted to the final volume of 50 μl with sterilized DW. The resultant was denatured at 94° C. for 5 min and then subject to PCR amplification for 30 cycles according to temperature parameters as follows: 94° C. for 30 sec. 58° C. for 30 sec and 72° C. for 2 min, and 72° C. for 5 min for final extension reaction. The PCR product was identified by electrophoresis with 1.5% agarose gel containing ethidium bromide for staining and was digested with XhoI and BamHI, followed by purification using GENECLEAN II kit (Bio101, USA). The purified fragment was denoted as “APC-b”.

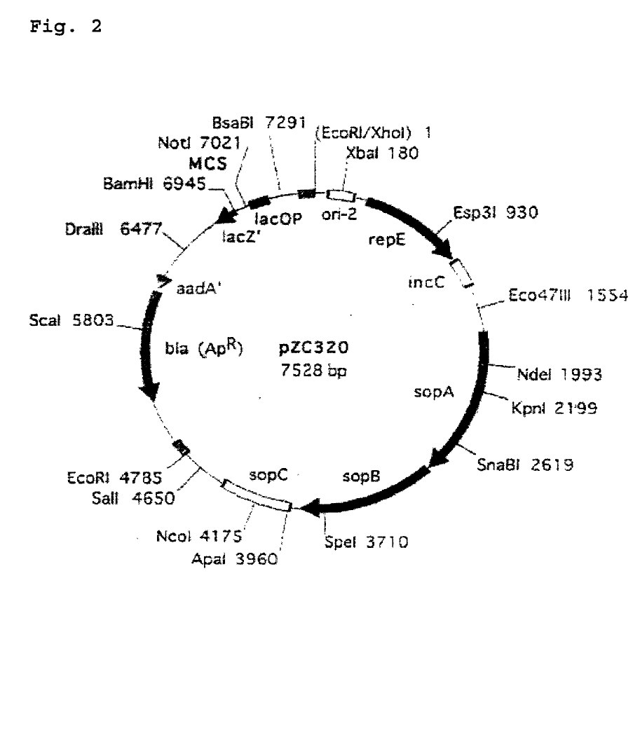

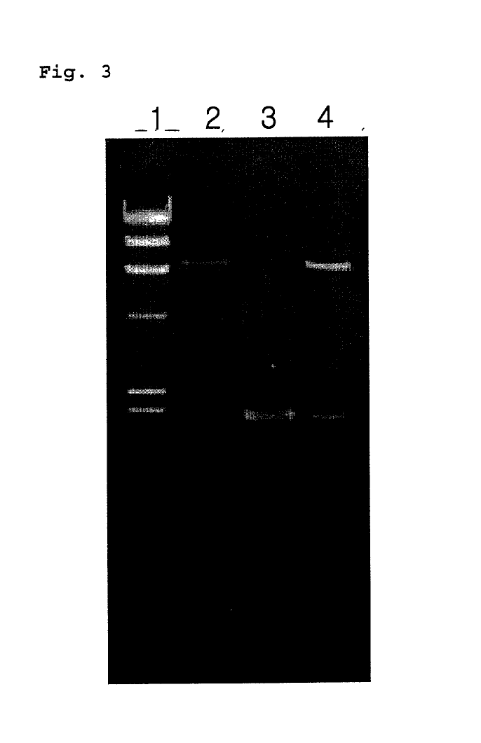

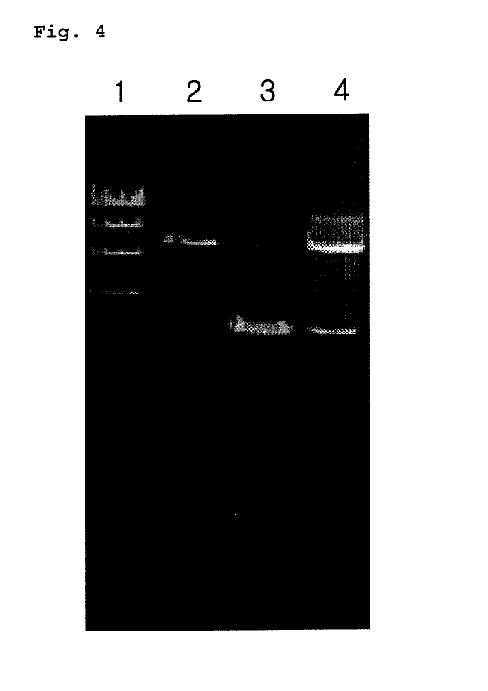

Thereafter, the multiple cloning site in lacZ locus of plasmid pZC320 (see FIG. 2; Jianpeng, S. et al., Gene, 146:55(1995)) which is a low copy-numbered plasmid for E. coli, was cleavaged at XhoI and BamHI sites and the resultant plasmid fragment was ligated to APC-a and APC-b., respectively. The recombinant plasmid was introduced into E. coli DH5α, followed by growth on medium containing X-gal and IPTG. The colonies with a blue color were selected and plasmid DNA was isolated therefrom. The plasmid carrying APC-a fragment was denoted as “pAPC-a” and the plasmid carrying APC-b fragment “pAPC-b”. Finally, the isolated plasmids pAPC-a and pAPC-b were digested with XhoI and BamHI and electrophoresed on 1.5% agarose gel, demonstrating the digested fragments (see FIGS. 3 and 4). In FIG. 3, lane 1 shows λ DNA molecular weight marker treated with HindIII; lane 2 plasmid pZC320 digested with XhoI and BamHI; lane 3 APC-a fragment; and lane 4 plasmid pAPC-a digested with XhoI and BamHI. In FIG. 4, lane 1 represents lane 1 shows λ DNA molecular weight marker treated with HindIII; lane 2 plasmid pZC320 digested with XhoI and BamHI; lane 3 APC-b fragment; and lane 4 plasmid pAPC-b digested with XhoI and BamHI. As shown in FIGS. 3 and 4, the fragments, both APC-a and APC-b, were successfully cloned into pZC320 with a low copy number for E. coli.

I-2: Construction of Gap Vector for APC

Carrying out PCR using pAPC-a and pAPC-b as templates obtained from Example I-1, the gap vector carrying nucleotides 19-153 of APC gene at 5′ end and nucleotides 1828-1977 of APC gene at 3′ end and the gap vector carrying nucleotides 1978-2163 of APC gene at 5′ end and nucleotides 5089-5266 of APC gene at 3′ end were constructed as follows: The primers AV-a5 (see SEQ ID NO: 5) and AV-a3 (see SEQ ID NO: 6) were synthesized for PCR using pAPC-a as template and the primers AV-b5 (see SEQ ID NO: 7) and AV-b3 (see SEQ ID NO: 8) were synthesized for PCR using pAPC-b as template. Then, in the case of PCR using pAPC-a as template, pAPC-a 1 μg, 10X reaction buffer 5 μl, primers AV-a5 and AV-a3 of 20 pmol, respectively, and 2.5 mM dNTP mixture 8 μl were adjusted to the final volume of 47.75 μl with sterilized DW. Following denaturation of the reactant at 98° C. for 5 min, PCR amplification was performed for 30 cycles with 2.5 units of Ex. Taq polymerse (Takara, Japan) according to temperature parameters as follows: 94° C. for 30 sec, 58° C. for 30 sec and 72° C. for 3 min, and 72° C. for 10 min for final extension reaction.

In the case of PCR using pAPC-b as template, PCR was performed in the same manner as PCR using pAPC-a, except for using pAPC-b as template and AV-b5 and AV-b3 as primers.

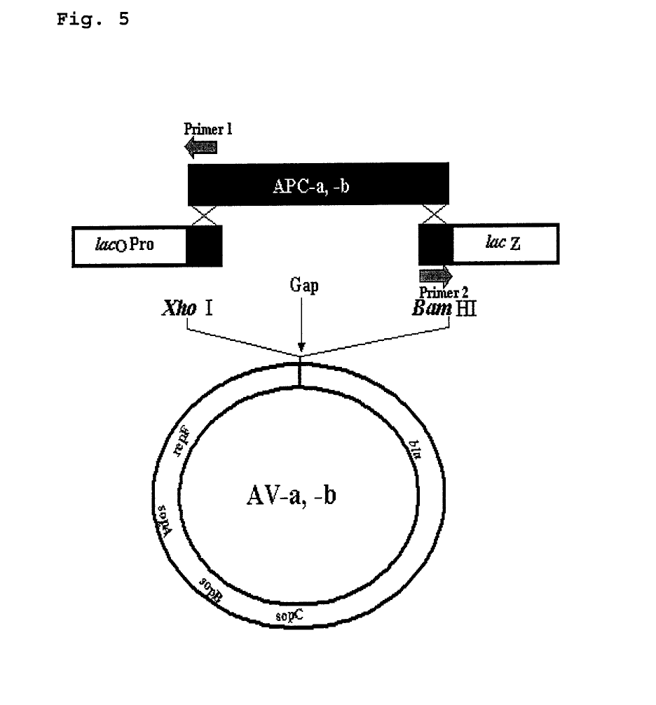

The gap vectors of two types were finally constructed, denoted as “AV-a” derived from pAPC-a and “AV-b” derived from pAPC-b (see FIG. 5). FIG. 5 shows the gap vectors, AV-a and AV-b, useful for ESC screening of APC gene mutation.

### EXAMPLE II-1

### Construction of Gap Vector for ESC Screening of BRCA1 Gene Mutation

II-1: BRCA1 Gene Cloning in Vector with a Low Copy Number for E. Coli

To construct plasmid employed as a template in PCR which is carried out in the construction of gap vector for ESC screening of BRCA1 gene, three fragments spanning nucleotides 99-903, nucleotides 789-4217 and nucleotides 4089-5704 of BRCA1 gene (GeneBank Accession No. U14680), corresponding to its exons 2-10, exon 11 and exon 12-24, respectively, were cloned into plasmid pZC320 with a low copy number for E. coli as follows:

The primers BRCA1-a5Xh (see SEQ ID NO: 9) and BRCA1-a3H (see SEQ ID NO: 10) were synthesized for cloning of nucleotides 104-904 of BRCA1 gene corresponding to its exons 2-10 in pZC320 with a low copy number for E. coli. In addition, the total cellular RNA was isolated from HBL-100 cell line (ATCC, HTB-124. USA) and its cDNA was amplified by RT-PCR as follows: To cDNA derived from the total cellular RNA above 20 μl, 25 mM MgCl₂ 6 μl, 10X LA PCR buffer II 8 μl, sterilized water 65.1 μl, TaKaRa Ex Taq 0.5 μl, and 10 pmol primers BRCA1-a5Xh and BRCA1-a3H of 2 μl, respectively were added, well mixed, subject to denaturation at 94° C. for 5 min and then to PCR amplification for 30 cycles according to temperature parameters as follows: 94° C. for 30 sec, 55° C. for 30 sec and 72° C. for 30 sec. The amplified product was identified by electrophoresis on 1.5% agarose gel containing ethidium bromide for staining and was digested with XhoI and BamHI, followed by purification using GENECLEAN II kit (Bio101, USA). The purified fragment was denoted as “BRCA1-a”.

The primers BRCA1-b5Xh (see SEQ ID NO: 11) and BRCA1-b3H (see SEQ ID NO: 12) were synthesized for cloning of nucleotides 789-4217 of BRCA1 gene corresponding to its exon 11 in pZC320 vector with a low copy number for E. coli. In addition, gDNA was isolated from HBL-100 cell line and amplified by PCR as follows: The gDNA as template 1 μg, 10X reaction buffer (100 mM Tris-HCl(pH 8.3), 500 mM KCl, 15 mM MgCl₂) 5 μl, primers BRCA1-b5Xh and BRCA1-b3H of 20 pmol, respectively and Ex. Taq polymerse (Takara, Japan) 2.5 unit were adjusted to the final volume of 50 μl with sterilized DW. The resultant was denatured at 94° C. for 5 min and then subject to PCR amplification for 30 cycles according to temperature parameters as follows: 94° C. for 30 sec, 55° C. for 30 sec and 72° C. for 1 min and 30 sec, and 72° C. for 5 min for final extension reaction. The amplified product was identified by electrophoresis with 1.5% agarose gel containing ethidium bromide for staining and was digested with XhoI and BamHI, followed by purification using GENECLEAN II kit (Bio100, USA). The purified fragment was denoted as “BRCA1-b”.

Moreover, for cloning of nucleotides 4089-5704 of BRCA1 gene corresponding to its exons 12-24 in pZC320 vector, RT-PCR was performed for 30 cycles as above. The primers used were BRCA1-c5Xh (see SEQ ID NO: 13) and BRCA1-c3H (see SEQ ID NO: 14). The initial denaturation reaction was carried out at 94° C. for 5 min. The temperature parameters for PCR are as follows: 94° C. for 30 sec, 55° C. for 30 sec and 72° C. for 1 min and 30 sec. The amplified product was identified by electrophoresis with 1.5% agarose gel containing ethidium bromide for staining and was digested with XhoI and BamHI, followed by purification using GENECLEAN II kit (Bio101, USA). The purified fragment was referred to as “BRCA1-c”.

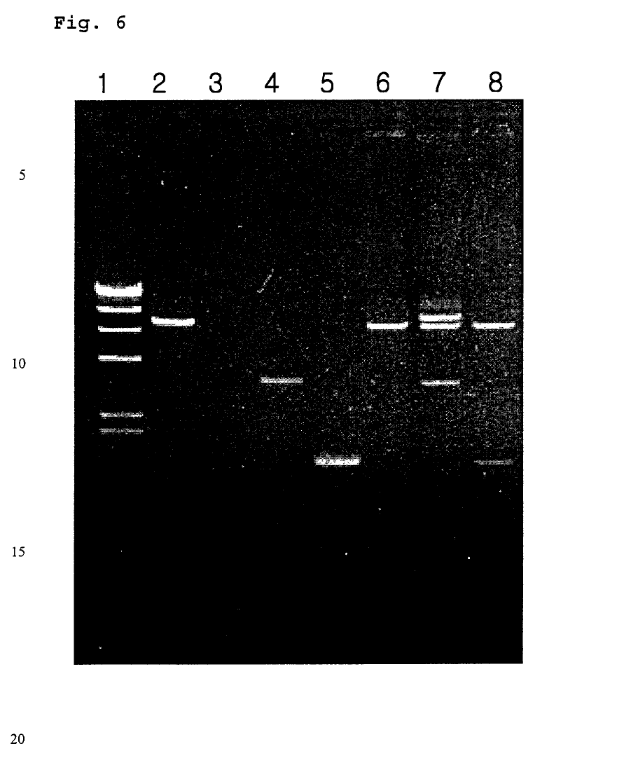

Thereafter, the multiple cloning site in lacZ locus of plasmid pZC320 was cleavaged at XhoI and BamHI sites and the resulting plasmid fragment was ligated to BRCA1-a, BRCA1-b and BRCA1-c, respectively. The recombinant plasmid was introduced into E. coli DH5α, followed by growth on medium containing X-gal and IPTG. The colonies with a blue color were selected and plasmid DNA was isolated therefrom. The plasmid carrying BRCA1-a fragment was denoted as “pBRCA1-a”, the plasmid carrying BRCA1-b fragment “pBRCA1-b” and the plasmid carrying BRCA1-c fragment “pBRCA1-c”. Finally, each of the isolated plasmids was digested with XhoI and BamHI and electrophoresed on 1.5% agarose gel, demonstrating the digested fragments (see FIG. 6). In FIG. 6, lane 1 shows λ DNA molecular weight marker treated with HindIII; lane 2 plasmid pZC320 digested with XhoI and BamHI; lane 3 BRCA1-a fragment; lane 4 BRCA1-b fragment; lane 5 BRCA1-c fragment; lane 6 plasmid pBRCA1-a digested with XhoI and BamHI; lane 7 plasmid pBRCA1-b digested with XhoI and BamHI; and lane 8 plasmid pBRCA1-c digested with XhoI and BamHI. As shown in FIG. 6, the fragments, BRCA1-a, BRCA1-b and BRCA1-c were all successfully cloned into pZC320 with a low copy number for E. coli.

II-2: Construction of Gap Vector for BRCA1

Carrying out PCR using pBRCA1-a, pBRCA1-b and pBRCA1-c as templates from Example II-1, the gap vector carrying nucleotides 99-203 of BRCA1 gene at 5′ end and nucleotides 798-903 of BRCA1 gene at 3′ end; the gap vector carrying nucleotides 789-932 of BRCA1 gene at 5′ end and nucleotides 4063-4217 of BRCA1 gene at 3′ end; and the gap vector carrying nucleotides 4089-4217 of BRCA1 gene at 5′ end and nucleotides 5619-5704 of BRCA1 gene at 3′ end were constructed as follows: The primers BV-a5 (see SEQ ID NO: 15) and BV-a3 (see SEQ ID NO: 16) were synthesized for PCR using pBRCA1-a as template, the primers BV-b5 (see SEQ ID NO: 17) and BV-b3 (see SEQ ID NO: 18) were synthesized for PCR using pBRCA1-b as template and the primers BV-c5 (see SEQ ID NO: 19) and BV-c3 (see SEQ ID NO: 20) were synthesized for PCR using pBRCA1-c as template. Then, the template DNA 1 μg, a pair of primers of 20 pmol, respectively, 10X reaction buffer 5 μl and 2.5 mM dNTP mixture 8 μl were adjusted to the final volume of 47.75 μl with sterilized DW. Following denaturation of the reactant at 98° C. for 5 min, PCR amplification was performed for 30 cycles with 2.5 units of Ex. Taq polymerse (Takara, Japan) according to temperature parameters as follows: 94° C. for 30 sec, 58° C. for 30 sec and 72° C. for 3 min, and 72° C. for 10 min for final extension reaction.

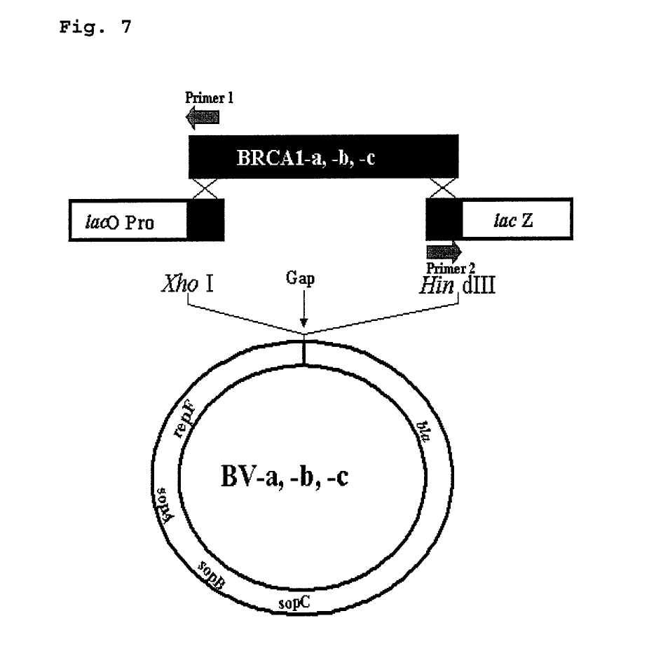

The gap vectors of three types were finally constructed, denoted as “BV-a” derived from pBRCA1-a, “BV-b” derived from pBRCA1-b and “BV-c” derived from pBRCA1-c (see FIG. 7). FIG. 7 shows the gap vectors, BV-a, BV-b and BV-c, useful for ESC screening of BRCA1 gene mutation.

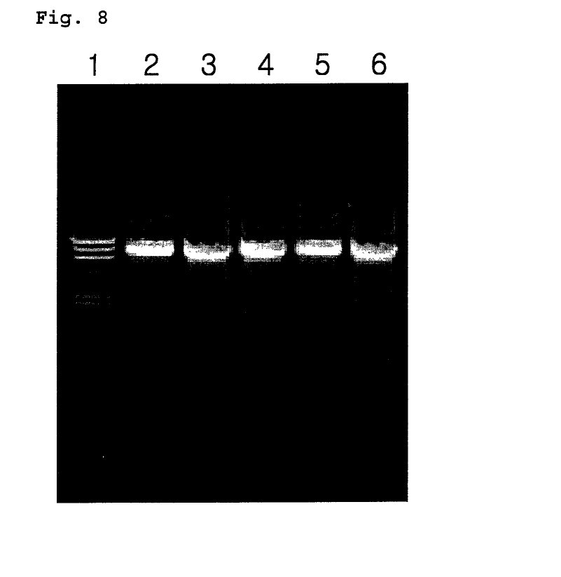

The gap vectors constructed in Examples I and II were electrophoresed on agarose gel and the band patterns are represented in FIG. 8. In FIG. 8, lane 1 shows λ DNA molecular weight marker treated with HindIII; lane 2 gap vector AV-a; lane 3 gap vector AV-b; lane 4 gap vector BV-a; lane 5 gap vector BV-b; and lane 6 gap vector BV-c.

### EXAMPLE III

### Determination on Heterozygous Mutation of APC gene Using Gap Vector

III-1: Amplification of APC Gene

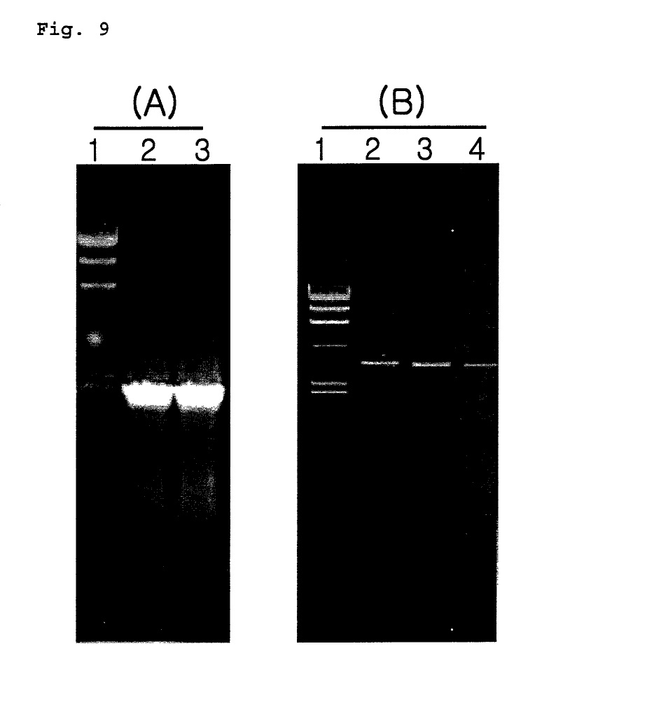

The PCR amplification of fragments of APC-a and APC-b for ESC was performed as follows: The primers APC-a5Xh and APC-a3Ba for PCR of APC-a fragment corresponding to the exons 1-14 of APC gene and the primers APC-b5 (see SEQ ID NO: 21) and APC-b3 for PCR of APC-b fragment corresponding to the exon 15 (see SEQ ID NO: 22) were synthesized. The templates for PCR of APC-a fragment were cDNAs from RT-PCR using RNAs isolated from HCT116 cell line and SW480 cell line (ATCC, CCL-228. USA) corresponding to Gardener's Syndrome (GS) family and familial adenomatous polyposis (FAP) family, respectively. The templates for PCR of APC-b fragment were gDNAs isolated from HCT116 cell line, SW480 cell line and peripheral blood sample of a normal individual. The electrophoresis on 1.5% agarose gel of the products indicated the successful amplification (FIG. 9). In FIG. 9, lane 1 of panel (A) shows λ DNA molecular weight marker treated with HindIII, lane 2 APC-a fragment amplified with cDNA from HCT116 cell line, and lane 3 APC-a fragment amplified with cDNA from SW480 cell line; and lane 1 of panel (B) shows λ DNA molecular weight marker treated with HindIII, lane 2 APC-b fragment amplified with gDNA from HCT116 cell line, lane 3 APC-b fragment amplified with gDNA from SW480 cell line, and lane 4 APC-b fragment amplified with gDNA from the peripheral blood sample of a normal individual. As indicated in FIG. 9, APC-a fragment was successfully amplified in size of about 1.9 kb by RT-PCR using RNA from HCT116 cell line and SW480 cell line. APC-b fragment was also successfully amplified in the size of about 3.3 kb using gDNAs from the above samples.

Consequently, it is noted that the present primers particularly employed in amplification of certain exon regions are also good for PCR-amplification of fragments of APC gene originated from a variety of samples.

III-2: Cotransformation

The DNA fragments yielded in Example III-1 and each of gap vectors were introduced into E. coli for inducing gap repair by homologous recombination, finally performing ESC screening as follows: Competent E. coli DH5α cells were cotransformed with 2 μl of the PCR fragment APC-a and 1 μl of gap vector AV-a or 2 g of the PCR fragment APC-b and 1 μl of the gap vector AV-b, thereby inducing gap repair. Transformants were plated on solid LB medium containing X-gal and IPTG following incubation at 37° C. for 14-16 hrs. The number and the color of colonies generated were counted.

TABLE 1
Evaluation on gap repair by cotransformation of
gap vector and DNA fragment

| | | | | Ratio of |
|---|---|---|---|---|
| Gap | | Number of Colony | | Blue |
| Vector | Insert | Blue Colony | White Colony | Colonies (%) |
| AV-a | — | 0 | 0 | |
| | APC-a | 31 | 0 | 100 |
| AV-b | — | 0 | 1 | |
| | APC-b | 65 | 0 | 100 |

As demonstrated in Table 1, E. coli cells cotransformed with both gap vector and DNA fragment show grown colonies with a blue color; but E. coli cells transformed only with gap vector show no colonies.

III-3: ESC Screening of Gardener's Syndrome Family

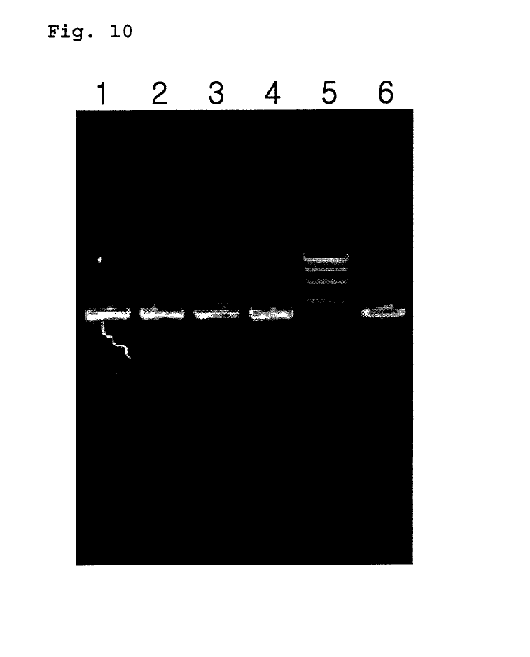

ESC screening was carried out for the members of gardener's syndrome family: a father (GSI-1) and three offspring (GSII-1, GSII-2 and GSII-3). gDNAs were isolated from peripheral blood samples of the members of GS family and PCR using primers APC-b5 and APC-b3, followed by agarose gel electrophoresis of amplified products (FIG. 10). In FIG. 10, lane 1 shows APC-b fragment amplified with gDNA from the peripheral blood of the father (GSI-l); lanes 2-4 APC-b fragment amplified with gDNA from peripheral blood of the offspring (GSII-1, GSII-2 and GSII-3), respectively; lane 5 λ DNA molecular weight marker treated with HindIII; and lane 6 APC-b fragment amplified with gDNA from the peripheral blood of a normal individual. As indicated in FIG. 10, APC-b fragments of approximately 3.3 kb were amplified from all the above samples by PCR.

Thereafter, competent E. coli DH5α cells were cotransformed with each of APC-b fragments amplified from samples above and gap vector AV-b, allowing gap repair. Transformants were plated on solid LB medium containing X-gal and IPTG following incubation at 37° C. for 14-16 hrs. The number and the color of colonies generated were observed (see Table 2).

TABLE 2
ESC screening APC gene defects of gardener's syndrome family

| | |
|---|---|
| Number of Colony | Ratio of Blue |

| Sample | Blue Colony | White Colony | Colonies (%) |
|---|---|---|---|
| Normal | 55 | 0 | 100 |
| GSI-1 | 24 | 20 | 54.5 |
| GSII-1 | 52 | 7 | 88.1 |
| GSII-2 | 26 | 16 | 61.9 |
| GSII-3 | 24 | 23 | 51.1 |

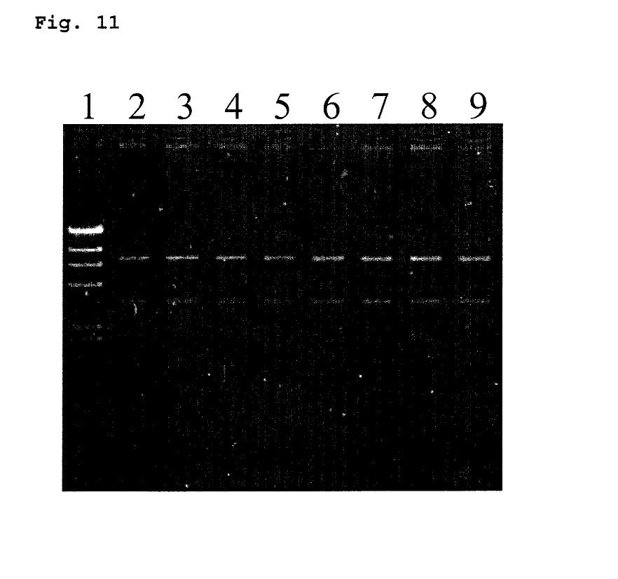

As demonstrated in Table 2, analysis of the number and the color of colonies shows two types of colonies: blue colonies and white colonies. The white colonies were not observed in the sample of a normal individual. Furthermore, to identify gap repair by homologous recombination in colonies generated from cotransformation of APC-b fragment and AV-b gap vector, plasmid DNA was isolated from colonies and digested with XhoI and BamHI, after which agarose electrophoresis was performed (see FIG. 11). In FIG. 11, lane 1 represents X DNA molecular weight marker treated with HindIII; lanes 2-5 plasmid DNA isolated from blue colonies of each sample and then digested with XhoI and BamHI; and lanes 6-9 plasmid DNA isolated from white colonies of each sample and then digested with XhoI and BamHI. As demonstrated in FIG. 11, all lanes show DNA fragments generated through digestion with restriction enzymes, indicating all colonies harbor recombinant plasmid.

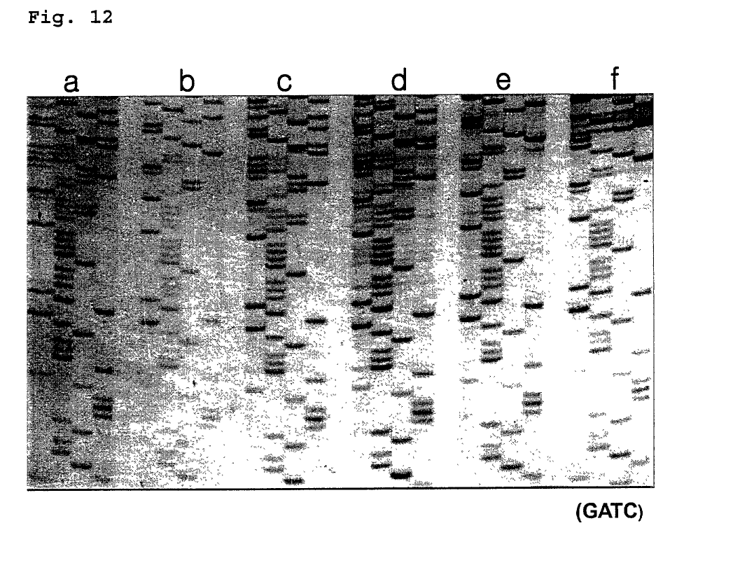

On the basis of the above results, to find the existence of heterozygous mutation in gDNA obtained from each sample, the primers Seq-F (see SEQ ID NO: 23) and Seq-R (see SEQ ID NO: 24) were synthesized. PCR amplification was performed with the primers and gDNA from each sample as template, thus amplifying mutation region (197 bp) corresponding to exon 15 of APC gene. With the amplified product as template, base sequencing was performed using Top™ Sequencing Kit (Bioneer, Korea) (see FIG. 12). In FIG. 12, panels (a)-(f) represent the base sequences of the amplified 197 bp from GSI-1, GSII-1, GSII-2, GSII-3, blue colonies and white colonies, respectively. As known in FIG. 12, heterozygous mutations are shown in GSI-1, GSII-2 and GSII-3; but not in GSII-1. The results demonstrate the reliability of the ESC assay of this invention designed for assay of heterozygous mutation.

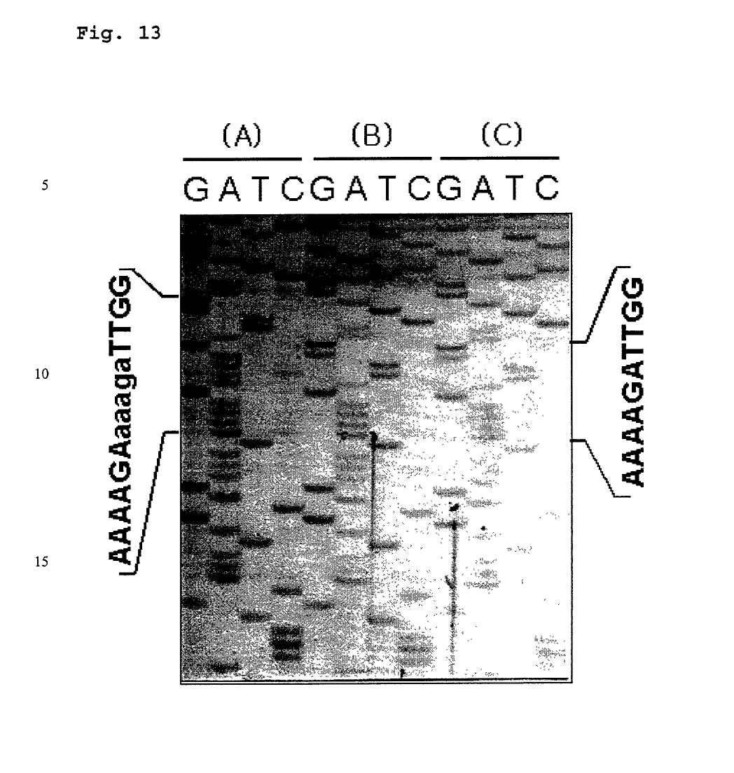

Subsequently, to identify that each allele corresponding to wild type and heterozygous mutation are separated precisely between blue colonies and white colonies, DNA was extracted from both blue colonies and white colonies and then sequenced (see FIG. 13) as described above. In FIG. 13, panel (a) represents the result of base sequencing of the amplified 197 bp from blue colonies and panels (b)-(c) the results of base sequencing of the amplified 197 bp from white colonies. As shown in FIG. 13, all of the recombinant DNAs isolated from blue colonies show APC-b fragments with normal base sequence, whereas the recombinant DNAs isolated from white colonies show APC-b fragments with 5 bp (GAAAA) deletion corresponding to codons 1309-1311. Therefore, it is identified that the deletion mutation leads to frameshift mutation, finally generating a stop codon.

Consequently, according to the results as described, in GS family, the father (GSI-1) and two offspring (GSII-2 and GSII-3) show heterozygous mutation in APC-b fragment of APC gene, which were confirmed by repeated experiments. Hence, the ESC assay of this invention offers a rapid and easy method for assaying truncating mutations in certain gene only through examination of colony color.

### EXAMPLE IV

### Determination on Heterozygous Mutation of BRCA1 Gene Using Gap Vector

IV-1: Amplification of BRCA1 Gene

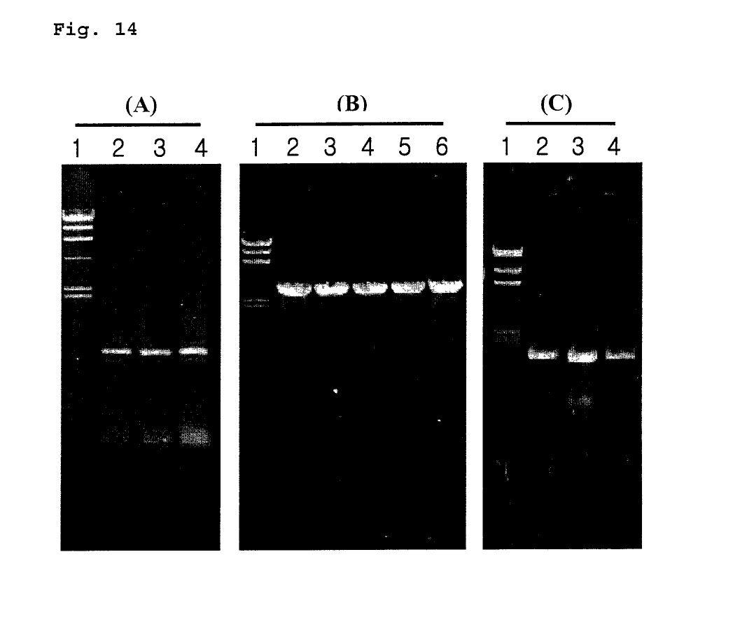

The PCR amplification of fragments of BRCA1-a and BRCA1-b for ESC was performed as follows: The primers BRCA1-a5Xh and BRCA1-a3H for PCR of BRCA1-a fragment corresponding to the exons 2-10 of BRCA1 gene, the primers BRCA1-b5Xh and BRCA1-b3H for PCR of BRCA1-b fragment corresponding to the exon 11 and the primers BRCA1-c5Xh and BRCA1-c3H for PCR of BRCA1-c fragment corresponding to the exons 12-24 were synthesized. The templates for PCR of each DNA fragment was gDNA or cDNA by RT-PCR derived from HBL-100 cell line, HCC1937 cell line(ATCC, No. CRL-2336. USA) and peripheral blood samples of normal individuals. The PCR was performed as described in Example I followed by 1.5% agarose gel electrophoresis (see FIG. 14). In FIG. 14, lane 1 of panel (A) shows λ DNA molecular weight marker treated with HindIII and lanes 2-4 BRCA1-a fragments amplified from HBL-100, HCC1937 and peripheral blood sample of normal individual, respectively; lane 1 of panel (B) shows λ DNA molecular weight marker treated with HindIII, lanes 2 and 3 BRCA1-a fragments amplified from HBL-100 and HCC1937, respectively, lanes 4-6 BRCA1-a fragments amplified from peripheral blood samples of normal individuals; and lane 1 of panel (c) shows A DNA molecular weight marker treated with HindIII and lanes 2-4 BRCA1-a fragments amplified from HBL-100, HCC1937 and peripheral blood samples of normal individuals, respectively. As shown in FIG. 14, each of BRCA1 DNA fragments was successfully amplified. Consequently, it is confirmed that the primers of this invention particularly employed in amplification of certain exon regions are also excellent for PCR-amplification of fragments of BRCA1 gene originated from a wide variety of samples.

IV-2: Cotransformation

The DNA fragments yielded in Example IV-1 and each of gap vectors were introduced into E. coli for inducing gap repair by homologous recombination, finally performing ESC assay as follows: Competent E. coli DH5α cells were cotransformed with 2 μl of fragment BRCA1-a and 1 μl of gap vector BV-a, 2 μl of fragment BRCA1-b and 1 μl of gap vector BV-b or 2 μl of fragment BRCA1-c and 1 μl of gap vector BV-c, thereby inducing gap repair. Transformants were plated on solid LB medium containing X-gal and IPTG following incubation at 37° C. for 14-16 hrs. The number and the color of colonies generated were examined (see Table 3).

TABLE 3
Evaluation on gap repair by cotransformation of
gap vector and DNA fragment

| | | | | Ratio of |
|---|---|---|---|---|
| Gap | | Number of Colony | | Blue |
| Vector | Insert | Blue Colony | White Colony | Colonies (%) |
| BV-a | — | 2 | 1 | — |
| | HBL-100 | 78 | 2 | 97.5 |
| BV-b | — | 1 | 1 | — |
| | HBL-100 | 43 | 6 | 87.8 |
| BV-c | — | 0 | 0 | — |
| | HBL-100 | 48 | 6 | 88.9 |

As demonstrated in Table 3, E. coli cells cotransformed with both gap vector and DNA fragment show grown colonies with a blue color; but E. coli cells transformed only with gap vector do not.

IV-3: ESC Assay of Breast Cancer Cell line

The reliability of ESC assay according to this invention was evaluated using HCC1937, breast cancer cell line, which have been identified to have mutation region at 3′ end of BRCA1 gene as follows: Competent E. coli DH5α cells were cotransformed with BRCA1-c fragment amplified from HCC1937 cell line and gap vector BV-c, allowing gap repair. Transformants were plated on solid LB medium containing X-gal and IPTG following incubation at 37° C. for 14-16 hrs. The number and the color of colonies generated were observed (see Table 4).

TABLE 4
ESC Assay for BRCA1 gene defects of breast cancer cell line

| | | | | Ratio of |
|---|---|---|---|---|
| Gap | | Number of Colony | | Blue |
| Vector | Insert | Blue Colony | White Colony | Colonies (%) |
| BV-c | — | 2 | 0 | — |
| | HCC1937 | 34 | 28 | 54.8 |

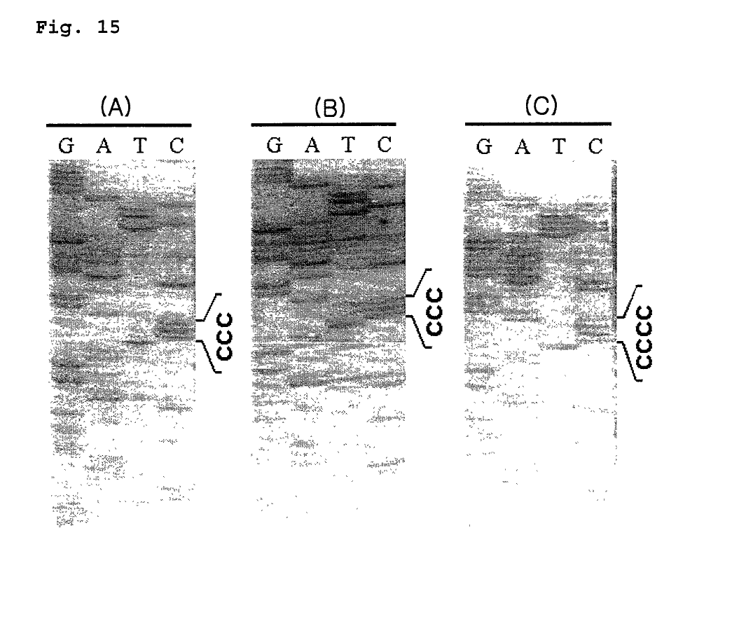

As demonstrated in Table 4, the examination of the number and the color of colonies distinctly shows two types of colonies: blue colonies and white colonies. To evaluate a reliability of the ESC assay of this invention in detecting heterozygous mutations, plasmid DNAs were isolated from each type of colony and sequenced using primer BR-Seq (see SEQ ID NO: 25) (see FIG. 15). In FIG. 15, panel (A) represents the base sequence of BRCA1-c fragments amplified from peripheral blood samples of normal individuals; and panels (B)-(C) the base sequence of BRCA1-c fragments plasmids isolated from blue colonies and white colonies, respectively. As known in FIG. 15, the base sequence of BRCA1-c fragment isolated from blue colonies and white colonies shows insertion of base C, whereas the other samples do not show it, which is well consistent with the results of the ESC assay of this invention as described. As confirmed in nucleotide sequencing, the ESC assay of this invention provides a reliable and efficient method for detecting heterozygous mutations in BRCA1 as well as in APC.

Having described a the preferred embodiments of the present invention, it is to be understood that variants and modifications thereof falling within the spirit of the invention may become apparent to those skilled in this art, and the scope of this invention is to be determined by appended claims and their equivalents.

What is claimed is:

1. A method for detecting heterozygous mutation on a gene of interest, which comprises the steps of:
(a) amplifying exon(s) of the gene of interest which is/are susceptible to truncating mutation;
(b) cloning the amplified exon(s) into plasmid with a low copy number for E. coli;
(c) performing PCR with the plasmid as template carrying the exon(s) and a set of primers designed to produce a gap vector having nucleotides of 50-200 bp at 5′ end and 3′ end thereof, respectively, corresponding to nucleotides at 5′ end and 3′ end of the amplified exon(s), respectively;
(d) isolating RNA or gDNA from specimen and amplifying DNA fragment by RT-PCR or PCR corresponding to the amplified exon(s);
(e) cotransforming E. coli with the gap vector and the amplified DNA fragment of (d); and
(f) determining the occurrence of gap repair of the gap vector of (e), wherein the gap repair is ascribed to homologous recombination between nucleotides at 5′ end and 3′ end of the gap vector and the cotransformed DNA fragment.

2. The method according to claim 1, wherein the gene of interest is APC gene or BRCA1 gene.

3. The method according to claim 1, wherein the gene of interest is APC gene and the set of primers designed to produce the gap vector are oligonucleotides consisting of the nucleotide sequence represented by SEQ ID NO:1 and SEQ ID NO:2 designed for amplifying exon 1 to exon 14 of APC gene.

4. The method according to claim 1, wherein the gene of interest is APC gene and the set of primers designed to produce the gap vector are oligonucleotides consisting of the nucleotide sequence represented by SEQ ID NO:3 and SEQ ID NO:4 designed for amplifying exon 15 of APC gene.

5. The method according to claim 1, wherein the gene of interest is BRCA1 gene and the set of primers designed to produce the gap vector are oligonucleotides consisting of the nucleotide sequence represented by SEQ ID NO:9 and SEQ ID NO:10 designed for amplifying exon 2 to exon 10 of BRCA1 gene.

6. The method according to claim 1, wherein the gene of interest is ERCA1 gene and the set of primers designed to produce the gap vector are oligonucleotides consisting of the nucleotide sequence represented by SEQ ID NO:11 and SEQ ID NO:12 designed for amplifying exon 11 of BRCA1 gene.

7. The method according to claim 1, wherein the gene of interest is BRCA1 gene and the set of primers designed to produce the gap vector are oligonucleotides consisting of the nucleotide sequence represented by SEQ ID NO:13 and SEQ ID NO:14 designed for amplifying exon 12 to exon 24 of BRCA1 gene.

8. The method according to claim 1, wherein the gap vector carries a lacZ gene as selective marker to determine the occurrence of the gap repair.

9. The method according to claim 8, wherein the step (f) is performed by incubating the cotransformed E. coli in medium containing X-gal and IPTG.

10. The method according to claim 1, wherein the plasmid for E. coli has a replication origin derived from F plasmid.

11. The method according to claim 10, wherein the replication origin is ori-2.

12. A gap vector for E. coli stop codon assay comprising:
(a) DNA fragment of a gene of interest located at 5′ end of the gap vector in which the gene of interest is susceptible to truncating mutation;
(b) DNA fragment of the gene of interest located at 3′ end of the gap vector in which the DNA fragment has a different sequence from the DNA fragment of (a);
(c) lacZ gene downstream of the DNA fragment of (a);
(d) lac operator and promoter upstream of the DNA fragment of (b) to control the expression of the DNA fragments and lacZ gene; and
(e) replication origin ensuring the copy number of the gap vector to be 1-1.5; in which a gap exists between the DNA fragments (a) and (b), whereby a homologous recombination occurs between the DNA fragments and a gene of interest to lead to gap repair of the gap vector.

13. The gap vector according to claim 12, wherein the replication origin is derived from F plasmid.

14. The gap vector according to claim 13, wherein the replication origin is ori-2.

15. The gap vector according to claim 12, wherein the DNA fragments is 50-200 bp in size.

16. The gap vector according to claim 12, wherein the gene of interest is APC gene or BRCA1.

17. A gap vector for E. coli stop codon assay constructed by a process comprising the steps of:
(a) amplifying exon(s) of a gene of interest which is/are susceptible to truncating mutation;
(b) cloning the amplified exon(s) in plasmid with a low copy number for E. coli; and
(c) performing PCR with the plasmid carrying the exon(s) as template and a set of primers to produce a gap vector having nucleotides of 50-200 bp at 5′ end and 3′ end thereof, respectively, corresponding to nucleotides at 5′ end and 3′ end of the amplified exon(s), respectively; in which a gap is generated between the nucleotides located at 5′ end and at 3′ end.

18. The gap vector according to claim 17, wherein the plasmid for E. coli has a replication origin derived from F plasmid.

19. The gap vector according to claim 18, wherein the replication origin is ori-2.
